(19) United States
(12) Patent Application Publication
(10) Pub. No.: US 2024/0210402 A1
(43) Pub. Date: Jun. 27, 2024

(54) METHOD FOR DETECTING ORAL NEOPLASTIC LESION, TEST REAGENT, TEST KIT AND THERAPEUTIC COMPOSITION

(71) Applicant: KANSAI MEDICAL UNIVERSITY EDUCATIONAL CORPORATION, Hirakata-shi, Osaka (JP)

(72) Inventors: Yuri NODA, Hirakata-shi, Osaka (JP); Kouji TSUTA, Hirakata-shi, Osaka (JP)

(21) Appl. No.: 18/557,658

(22) PCT Filed: Apr. 28, 2022
(86) PCT No.: PCT/JP2022/019344

§ 371 Date: Oct. 27, 2023

(30) Foreign Application Priority Data
Apr. 28, 2021 (JP) 2021-076213

(51) Int. Cl. G01N 33/574; A61K 39/395; G01N 33/68

(52) U.S. Cl. CPC G01N 33/57407; A61K 39/39558; G01N 33/6842

(57) ABSTRACT

The objective of the present invention is to provide the biomarker that enables carly finding and diagnosis of the oral cancer and precancerous lesion. This objective is reached with a method for detecting an oral neoplastic lesion comprising detecting phosphorylated mixed lineage kinase domain-like protein (MLKL) in an oral tissue or oral-derived cells collected from a subject.

### Technical Field

The present invention relates to a method for detecting an oral neoplastic lesion, a test reagent, a test kit and a therapeutic composition.

### Background Art

The number of patients with oral cancer in Japan has tripled over the past 30 years, with approximately 22,500 cases and over 7,900 deaths expected annually. However, the awareness of oral cancer and a precancerous lesion that occur on a tongue, gingiva, buccal mucosa, floor of the mouth, and palate remains low, and the 5-year survival rate is as low as 60%, because many are found in advanced stages due to delays in detection and diagnosis (Non Patent Literature 1). In addition, since the disease is detected in advanced stages, there are many cases in which extended surgery is indicated after its detection, and dysarthria, difficulty in eating and changes in facial appearance caused by surgical resection often interfere with subsequent life.

On the other hand, as to a phosphorylated mixed lineage kinase domain-like protein (p-MLKL), decreased expression of the p-MLKL in tumors has been found, at the protein level, to correlate with a worse prognosis and chemoresistance in colorectal (Non Patent Literature 2), cervical (Non Patent Literature 3), gastric (Non Patent Literature 4) and ovarian (Non Patent Literature 5) cancers. In addition, it has recently been reported that in oral squamous cell carcinoma, increased abundance of the p-MLKL results in reduced overall survival (OS) and progression-free survival (PFS) (Non Patent Literature 6).

### Citation List

### Non Patent Literature

Non Patent Literature 1: Mortality database No. of deaths-Malignant neoplasm of lip, oral cavity and pharynx, both sexes. WHO.

Non Patent Literature 2: Li X, et al. Technol Cancer Res Treat. 2017;16(4):428-434.

Non Patent Literature 3: Ruan J, et al. Int J Clin Exp Pathol. ; 8(11):15035-8.

Non Patent Literature 4: Sun W, et al. Oncol Lett. 2019 ; 18(4):3830-3836.

Non Patent Literature 5: He L, et al. Onco Targets Ther. 2013;6:1539-43.

Non Patent Literature 6: Li J, et al. Cell Death Dis. 2020; 11(5):391.

### SUMMARY OF INVENTION

### Technical Problem

In view of the current situation described above, early finding, appropriate diagnosis and early treatment of lesions are important to improve the QOL and survival rate of the oral cancer and precancerous lesion. However, at present, no biomarker has been found that enables early finding and diagnosis of the oral cancer and precancerous lesion.

An object of the present invention is to provide the biomarker that enables early finding and diagnosis of the oral cancer and precancerous lesion.

### Solution to Problem

As a result of diligent research, the present inventors have found that the abundance of phosphorylated mixed lineage kinase domain-like protein (MLKL) is increased in oral cancer and a precancerous lesion. The present invention has been achieved based on the above findings, and includes the following aspects.

Item 1. A method for detecting an oral neoplastic lesion comprising detecting phosphorylated mixed lineage kinase domain-like protein (MLKL) in an oral tissue or oral-derived cells collected from a subject.

Item 2. The method according to item 1, wherein the oral neoplastic lesion is a squamous cell carcinoma or dysplasia.

Item 3. The method according to item 2, wherein the squamous cell carcinoma is a well-differentiated squamous cell carcinoma.

Item 4. The method according to any one of items 1 to 3, wherein the oral tissue is an upper layer of squamous epithelium or the oral-derived cells are derived from the upper layer.

Item 5. A method for using phosphorylated mixed lineage kinase domain-like protein (MLKL) to detect an oral neoplastic lesion in an oral cavity.

Item 6. A test reagent for use in the method according to any one of items 1 to 4, comprising an anti-phosphorylated mixed lineage kinase domain-like protein (MLKL) antibody.

Item 7. A test kit for use in the method according to any one of items 1 to 4, comprising the test reagent according to item 6.

Item 8. A test reagent for detecting an oral neoplastic lesion for use in an oral cavity, comprising an anti-phosphorylated mixed lineage kinase domain-like protein (MLKL) antibody.

Item 9. A test kit for detecting an oral neoplastic lesion for use in an oral cavity, comprising the test reagent according to item 8.

Item 10. A therapeutic composition for treating an oral neoplastic lesion for use in photoimmunotherapy, comprising an anti-phosphorylated mixed lineage kinase domain-like protein (MLKL) antibody.

### Advantageous Effects of Invention

It can provide a biomarker that is highly specific for oral cancer and precancerous lesions. In addition, said biomarker can be a target for photoimmunotherapy and the like.

### BRIEF DESCRIPTION OF DRAWINGS

FIG. 1 shows images of an immunostained tissue for p-MLKL in a case of non-neoplastic lesion. Panel (a) shows results in an inflamed tissue. Panel (a) left is an image of an HE-stained tissue, and the figure on Panel (a) right is an image of an immunostained tissue for the p-MLKL. Panel (b) shows results in a hyperplastic tissue. Panel (b) left is an image of an HE-stained tissue, and Panel (b) right is an image of an immunostained tissue for the p-MLKL. Panel (c) shows a case in which the p-MLKL was positive in an inflammatory lesion. Panel (c) left is an image of an HE-stained tissue, and Panel (c) right is an image of an immunostained tissue for the p-MLKL.

FIG. 2 shows images of an immunostained tissue for the p-MLKL in a precancerous lesion (OED). Panel (a) shows results in a case of mild dysplasia. Panel (a) left is an image of an HE-stained tissue, and Panel (a) right is an image of an immunostained tissue for the p-MLKL. Panel (b) shows results in a case of moderate dysplasia. Panel (b) left is an image of an HE-stained tissue, and Panel (b) right is an image of an immunostained tissue for the p-MLKL. Panel (c) shows results in a case of severe dysplasia. Panel (c) left is an image of an HE-stained tissue, and Panel (c) right is an image of an immunostained tissue for the p-MLKL.

FIG. 3 shows images of an immunostained tissue for the p-MLKL in a squamous cell carcinoma. Panel (a) and Panel (b) show results of a tissue in which the p-MLKL signal was strongly positive. Panel (a) left and Panel (b) left are images of an HE-stained tissue, and Panel (a) right and Panel (b) right are images of an immunostained tissue for the p-MLKL. Panel (c) shows results of a tissue in which the p-MLKL signal was moderately positive. Panel (c) left is an image of an HE-stained tissue, and Panel (c) right is an image of an immunostained tissue for the p-MLKL.

FIG. 4 shows images of an immunostained tissue for the p-MLKL in the squamous cell carcinoma. Panel (a) shows results of a tissue in a case of well-differentiated squamous cell carcinoma. Panel (a) left is an image of an HE-stained tissue, and Panel (a) right is an image of an immunostained tissue for the p-MLKL. Panel (b) shows results of a tissue in a case of basaloid squamous cell carcinoma. Panel (b) left is an image of an HE-stained tissue, and Panel (b) right is an image of an immunostained tissue for the p-MLKL. Panel (c) shows results of a tissue in a case of poorly-differentiated squamous cell carcinoma in which the p-MLKL was positive. Panel (c) left is an image of an HE-stained tissue, and the figure on Panel (c) right is an image of an immunostained tissue for the p-MLKL.

FIG. 5 shows the positive rates of the p-MLKL in a non-neoplastic tissue, precancerous lesion (OED) and squamous cell carcinoma (OSCC). Numbers in the table indicate the “number of positives (positive rate (%))”.

FIG. 6 shows results of sensitivity, specificity, PPV and NPV for detection of an oral squamous cell carcinoma when the p-MLKL is used. Table (a) shows test accuracy when the p-MLKL was determined to be positive in an upper layer. Table (b) shows test accuracy when the p-MLKL was determined to be positive in either the upper layer or lower layer.

FIG. 7 shows results of sensitivity, specificity, PPV and NPV for detection of a dysplasia when the p-MLKL is used. Table (a) shows test accuracy when the p-MLKL was determined to be positive in an upper layer. Table (b) shows test accuracy when the p-MLKL was determined to be positive in either the upper layer or lower layer.

### DESCRIPTION OF EMBODIMENTS

### I. Method for Detecting Oral Neoplastic Lesion

One embodiment of the present invention relates to a method for detecting an oral neoplastic lesion. The detection method comprises detecting phosphorylated mixed lineage kinase domain-like protein (MLKL) in an oral tissue collected from a subject.

As used herein, the oral neoplastic lesion may include a squamous cell carcinoma, dysplasia that can be said to be a precancerous lesion thereof, non-neoplastic inflammation, and the like. In the detection method, the oral neoplastic lesion is preferably the squamous cell carcinoma or dysplasia. The squamous cell carcinoma may include a well-differentiated squamous cell carcinoma, moderately-differentiated squamous cell carcinoma and poorly-differentiated squamous cell carcinoma. The well-differentiated squamous cell carcinoma is more preferred as the squamous cell carcinoma. The dysplasia may include mild dysplasia, moderate dysplasia, and severe dysplasia. The moderate dysplasia and severe dysplasia are more preferred as the dysplasia.

The mixed lineage kinase domain-like protein (MLKL) is a protein found in cytoplasm and on membrane that plays an important role in an occurrence of necroptosis, a type of cell death involved in development of organisms and growth of cancers. A nonphosphorylated MLKL is phosphorylated in the cytoplasm through an inhibition of activity of apoptosis related factor caspase 8 by necroptosis stimulation. It has been reported that the phosphorylated MLKL (also referred to as the p-MLKL) is involved in the occurrence of necroptosis by translocating to cell membrane, forming membrane pores and facilitating entry of extracellular matrix into the cytoplasm. It has been reported that the 358th serine from an N-terminus (Ser 358) and the 345th serine from the N-terminus (Ser 345) are the phosphorylation sites of the MLKL. It is preferred herein to detect phosphorylation of the Ser 358 of the MLKL.

The subject may or may not be a patient suspected of having the oral neoplastic lesion.

The oral tissue collected from the subject is not limited as long as the p-MLKL can be detected. For example, tissue collected by biopsy, surgically resected tissue, tissue (cell population) contained in a scraped sample of oral mucosa, tissue (cell population) contained in a stamped sample of the oral tissue, tissue (cell population) contained in saliva, or the like may be used as the oral tissue. Furthermore, cells contained in the scraped sample of the oral mucosa, cells contained in the stamped sample of the oral mucosa, cells contained in the saliva, or the like may be used as the oral-derived cell. The scraped sample of the oral mucosa may be collected by directly scraping the inside of the subject's oral cavity with a cotton swab or microscope slide. The stamped sample of the oral tissue may be obtained by pressing biopsy or resected tissues against the microscope slide. The tissue contained in the saliva may be retrieved by centrifugation or filtering after the saliva is collected from the subject.

### (i) Detection of p-MLKL by Immunostaining

When the p-MLKL is detected by immunostaining, before the p-MLKL is detected, the biopsy or resected tissues are fixed with a known fixative solution such as formalin and paraformaldehyde and then paraffin-embedded blocks are prepared. Alternatively, the biopsy or resected tissues are fixed or unfixed and then embedded in a resin for preparing frozen blocks, such as O.C.T. Compound®, to prepare frozen blocks. The prepared paraffin-embedded or frozen block is thinly sliced to prepare tissue sections which is used for detection of the p-MLKL.

The tissue or orally-derived cell in the scraped sample, the stamped sample or the saliva may be used to detect the p-MLKL after the microscope slide to which they are attached is fixed with alcohol or the like.

Methods for detecting the p-MLKL are not limited as long as the p-MLKL can be detected in the oral tissue collected from the subject. The p-MLKL may be detected, for example, by the immunostaining.

The immunostaining may be performed using a known method. For example, the thin section prepared from the paraffin-embedded block is deparaffinized and subjected to a hydrophilic treatment, followed by blocking, in order to react with anti-p-MLKL antibody. If necessary, activation of antigen, inactivation of endogenous peroxidase or inactivation of endogenous alkaline phosphatase may be performed prior to the blocking. The activation of the antigen may be performed, for example, by immersing the sections in a citric acid buffer with a pH of about 5.8 to 9.0 at about 95° C. to 100° C. for 20 to 40 minutes. However, other methods may be used for this treatment as long as the antigen can be activated. The inactivation of the endogenous peroxidase is also known and may be performed using methanol or the like with hydrogen peroxide added. The inactivation of the endogenous alkaline phosphatase is also known and may be performed using alcohol with hydrochloric acid added or acetic acid solution.

In addition, the section prepared from the frozen block is washed with water or buffer to remove the resin for embedding, followed by blocking, and then reacted with anti-the p-MLKL antibodies. According to the method described above, the activation of the antigen, the inactivation of the endogenous peroxidase or the inactivation of the endogenous alkaline phosphatase may be performed prior to the blocking.

The tissue contained in the scraped sample, stamped sample or saliva is fixed, washed with the water or buffer to remove the resin for embedding, followed by blocking and reacted with the anti-p-MLKL antibody. According to the method described above, the activation of the antigen, the inactivation of the endogenous peroxidase or the inactivation of the endogenous alkaline phosphatase may be performed prior to the blocking.

Anti-MLKL (phospho S358) (Clone ID: EPR9514; catalog number: ab187091, RRID: AB_2619685; Abcam), Phospho-MLKL (Ser358) Polyclonal Antibody (PA5105678; Thermo Fisher), Phospho-MLKL (Ser358) (D6H3V) Rabbit mAb (#91689 Cell signaling), Phospho-MLKL (Ser345) Antibody (Mouse Specific) (#62233 Cell signaling), Phospho-MLKL (Ser358) (D6H3V) Rabbit mAb(#91689 Abcam), Anti-MLKL (phospho S358) antibody [EPR9514] (ab187091; Abcam), MLKL phospho (Ser358) antibody (ARG40184; arigo biolaboratories, Phospho-MLKL (Ser345) Antibody, clone 7C6.1 (Sigma Aldrich), MLKL (phospho Ser358) antibody (#GTX00973 Gene Tex), Anti-Phospho-MLKL (S358) Monoclonal Antibody (#MP00535 Boster Biological Technology), Anti-phospho-MLKL (S358) antibody (STJ97776) (St John's Laboratory Ltd), Human Phospho-MLKL (T357) Antibody (#MAB9187-SP R&D), Human Phospho-MLKL (T357) Antibody (#MAB9187-SP R&D), Recombinant Anti-MLKL (phospho S358) antibody [EPR9514] (ab187091; Abcam), Recombinant Anti-MLKL (phospho S345) antibody [EPR9515(2)] (ab196436; Abcam), Recombinant Rabbit Monoclonal Antibody Phospho-MLKL (Ser345) (JM92-37;ThermoFisher) or the like may be used for the immunostaining.

The immunostaining may be performed by a direct method or an indirect method. In the case of the direct method, the anti-p-MLKL antibody is directly labelled with a labelling substance such as a fluorescent substance, peroxidase or alkaline phosphatase, and the signal from these labelling substances is used as an indicator to detect the anti-p-MLKL antibody bound to the p-MLKL in the tissue on the microscope slide. In the case of the indirect method, an unlabelled anti-p-MLKL antibody (primary antibody) is reacted with the p-MLKL in the tissue on the microscope slide, washed with buffer or the like, and then reacted with a secondary antibody that can bind to the primary antibody. The secondary antibody is labelled with the labelling substance such as the fluorescent substance, peroxidase or alkaline phosphatase, and the signal from these labelling substances is used as the indicator to detect the anti-p-MLKL antibody bound to the p-MLKL in the tissue on the microscope slide.

When the labelling substance is a fluorescent dye, the p-MLKL in the tissue on the microscope slide is detected by observing a fluorescent signal emitted from the fluorescent dye using a fluorescence microscope.

When the labelling substance is the peroxidase, the p-MLKL is detected in the tissue on the microscope slide by observing the signal emitted form a substrate using the light microscope, for example, using diaminobenzidine (DAB) as the substrate.

When the labelling substance is the alkaline phosphatase, the p-MLKL is detected in the tissue on the microscope slide by observing the signal emitted from the substrate using the light microscope, for example, using New Fuchsin as the substrate.

After the immunostaining, counterstaining may be performed.

Whether the p-MLKL signal in the tissue is positive or negative may be determined by the following method.

For example, signal distribution area of the p-MLKL may be scored as “≤10%” or “>10%” in two fields of view. One field of view may be, for example, a 100x field of view (10x objective×10x eyepiece).

In addition, staining intensity may be scored as 0 (no signal), 1 (mild), 2 (moderate) or 3 (strong). Staining results may be defined as negative when the distribution area is ≤10% and the staining intensity is 0 or 1, and positive when the distribution area is >10% and the staining intensity is 2 or 3.

In addition, the squamous epithelial tissue in the oral cavity is composed of five layers from an oral side: stratum corneum, stratum intermedium or stratum granulosum, stratum spinosum, and stratum basale. The stratum corneum and stratum intermedium or stratum granulosum are classified as an upper layer, while the stratum spinosum and stratum basale are classified as a lower layer. Although the p-MLKL can appear in both the upper layer and the lower layer, it is preferable to detect the p-MLKL in the upper layer in this embodiment.

### (ii) Detection of p-MLKL by Immunoassay

The p-MLKL in the oral-derived cell collected from the subject may be detected by immunoassay, competitive immunoassay, or the like. The immunoassay may include Enzyme-Immuno assay (EIA) using an enzyme as the labelling substance, Fluorescence-Immuno assay (FIA) using the fluorescent substance as the labelling substance, Radio-immuno assay using a radioisotope as the labelling substance, and Lateral Flow Immunoassay utilizing an immunochromatography. EIA includes ELISA (Enzyme-Linked Immuno Sorbent Assay), in which an antigen is sandwiched between an antigen-capturing antibody and a detecting antibody and detected.

The antibody used for the immunoassay may be, for example, the antibody described in the above (i).

The specimen used for the immunoassay is not limited as long as it contains the oral-derived cell. The specimen may include the scraped sample for the immunoassay collected by scraping the inside of the oral cavity with the cotton swab or the like, the saliva, and the like. The scraped sample for the immunoassay and the saliva itself may be used as the specimen. Alternatively, the scraped sample for the immunoassay or the saliva with added a lysing agent that breaks down the cell membrane and mucus in the saliva to release the p-MLKL from the cell.

In the ELISA, the anti-p-MLKL antibody for capturing the antigen is immobilized on a solid phase such as a microplate, fluorescent beads or magnetic beads in advance, to form a complex of the immobilized anti-p-MLKL antibody and the p-MLKL in the specimen. By detecting the complex immobilized on the solid phase or the complex formed on the solid phase by methods known in the art, the p-MLKL in the specimen may be detected or the concentration of the p-MLKL may be measured. Alternatively, in this method, the complex of the anti-p-MLKL antibody for capturing the antigen and the p-MLKL in the specimen may be formed first, and then the complex may be immobilized on the solid phase.

The method of immobilizing the anti-p-MLKL antibody for capturing the antigen on the solid phase is not limited. It may be performed directly or indirectly via another substance using a known method. Methods of binding directly include, for example, physical adsorption. Specifically, each capture antibody may be directly physically bound to the microplate using an immunoplate or the like. Alternatively, the anti-p-MLKL antibody for capturing the antigen may be indirectly immobilized on the solid phase. The indirect immobilization of the capture antibody on fluorescent beads, magnetic beads, or the like, is known.

The form of the solid phase is not particularly limited, and includes, for example, the microplate, a microtube, a test tube, beads, a membrane, or the like. The material of the solid phase is not particularly limited, and for example, polystyrene, polypropylene, or the like may be used for the microplate, microtube, test tube, or the like. For the beads, polystyrene Xmap® beads (Luminex), MagPlex® microspheres (Luminex), or the like may be used. For the membrane, a nitrocellulose filter, nylon filter, paper, or the like may be used.

The present method may include an operation of washing the solid phase following the formation of said complex. For washing, PBS or the like containing a surfactant or the like may be used.

In the present method, in the case of a conventional immunoassay method, the detection of said complex may be performed using the anti-p-MLKL antibody for detection labeled with the labeling substance or using an anti-immunoglobulin antibody or the like labeled with the labeling substance capable of binding to the unlabeled anti-p-MLKL antibody. It is preferable to use the labeled anti-p-MLKL antibody for detection. In addition, it is preferable that an epitope on the antigen for the anti-p-MLKL antibody used for detection is different from the epitope on the antigen for the anti-p-MLKL antibody used for capturing the antigen.

When the p-MLKL is detected or the concentration of the p-MLKL is measured by the competitive immunoassay, the p-MLKL in the specimen is contacted with the anti-p-MLKL antibody for capture in the presence of a competitor labeled with the labeling substance. The competitor is a peptide or protein with the epitope that is recognized by the same capture antibody as the p-MLKL in the specimen. The competitor may be isolated from an individual, or synthesized by genetic engineering or chemically.

The labelling substance labelled on the anti-p-MLKL antibody, labelled anti-immunoglobulin antibody or competitor used for detection is not particularly limited, as long as a detectable signal is produced. The labelling substances may include, for example, a fluorescent substance, radioisotope, metal nanoparticle, enzyme, and the like The enzymes may include the alkaline phosphatase, peroxidase, and the like. The fluorescent substances may include a fluorescein isothiocyanate (FITC), rhodamine, fluorescent dye such as Alexa Fluor®, fluorescent protein such as GFP, and the like. The radioisotopes may include ¹²⁵I, ¹⁴C, ³²P and the like. The metal nanoparticles may include a gold nanoparticle and silver nanoparticle. Among these, the gold nanoparticle, alkaline phosphatase or peroxidase are preferred as the labelling substance.

The anti-p-MLKL antibody used for detection is obtained by labelling each antibody with the labelling substance described above using a labelling method known in the art. The labelling may be performed using a commercially available labelling kit or the like. The labelled immunoglobulin antibody may be labelled using the same technique as the labelling of the anti-p-MLKL antibody, or using the commercially available labelling kit.

In the present method, by detecting the signal produced by the labelling substance of the detecting antibody in the complex, the p-MLKL in the specimen may be detected or the concentration of the p-MLKL may be measured. Here, the phrase “detecting the signal” includes quantitatively detecting the presence or absence of the signal, quantifying the intensity of the signal, and semi-quantitatively detecting the intensity of the signal. Semi-quantitative detection refers to indicating the intensity of the signal in stages such as “no signal,” “weak,” “medium,” or “strong.” In the present method, it is preferable to detect the intensity of the signal quantitatively or semi-quantitatively.

A known method for detecting the signal may be used. In this method, the measurement method may be optionally selected depending on the type of signal derived from the above-mentioned labelling substance. For example, when the labeling substance is the enzyme, the signal such as light, color, or the like generated by reacting the substrate to said enzyme may be measured by visual observation, fluorometer, luminometer, spectrophotometer, or the like.

The substrate for the enzyme may be optionally selected from known substrates depending on the type of the enzyme. For example, when the alkaline phosphatase is used as the enzyme, the substrates may be selected from a chemiluminescent substrate such as CDP-Star® (Disodium 4-chloro-3-(methoxyspiro[1,2-dioxetane-3,2′-(5′-chloro)tricyclo[3.3.1.1^{3.7}]decan]-4-yl)phenyl phosphate) or the like, and a chromogenic substrate such as 5-bromo-4-chloro-3-indolyl phosphate (BCIP), 5-Bromo-6-chloro-3-indolyl phosphate disodium salt, and p-Nitrophenylphosphate or the like. When the labeling substance is the peroxidase, the substrates may include Tetramethylbenzidine (TMB) and the like.

When the labeling substance is the radioisotope, radiation as the signal may be measured using a known device such as a scintillation counter or the like. When the labeling substance is the fluorescent substance, fluorescence as the signal may be measured using a known device such as a fluorescent microplate reader, Luminex® system (Luminex) or the like. The excitation and fluorescence wavelengths may be determined appropriately depending on the type of the fluorescent substance used.

When the labeling substance is the gold nanoparticle, red color of the gold nanoparticles aggregated by an antigen-antibody reaction may be detected as the signal, or it may be detected by silver enhancement, in which silver ions are adsorbed on the gold nanoparticles in the presence of a reducing agent. The coloration by metal nanoparticles or by silver enhancement may be visually observed or its concentration may be measured optically. The immunoassay using the metal nanoparticles as the labeling substance is preferably performed by a lateral flow immunoassay in which, for example, a nitrocellulose filter is loaded with the capture antibody and detection antibody, and the capture antibody and detection antibody are reacted with the antigen in the specimen using the principle of chromatography.

The result of the signal detection may be used as the value of the concentration of the p-MLKL. For example, when the intensity of the signal is detected quantitatively, the measured value of the signal intensity itself or the value calculated from the measured value of the signal intensity may be used as the value of the p-MLKL concentration.

When the value of the concentration of the p-MLKL is used to determine whether the p-MLKL is detected or not, the determination may be performed based on a predetermined reference value. The predetermined reference value is not limited as long as it is a value that can most accurately determine whether p-MLKL is positive or negative. The “value that can classify most accurately” may be set appropriately based on an index such as the sensitivity, specificity, positive predictive value, negative predictive value, or the like, depending on the purpose of a test. The predetermined reference value may be the value of the concentration of the p-MLKL in persons who do not have an oral neoplastic lesion, an average value thereof, or the like.

When the value of the concentration of the p-MLKL in the subject is higher than the predetermined reference value, it means that the p-MLKL has been detected. When the value of the concentration of the p-MLKL in the subject is lower than the predetermined reference value, it means that the p-MLKL has not been detected.

When the value of the concentration of the p-MLKL in the subject is higher than the predetermined reference value, it can be determined that the subject has the oral neoplastic lesion. When the value of the concentration of the p-MLKL in the subject is lower than the predetermined reference value, it can be determined that the subject does not have the oral neoplastic lesion.

### II. Method for using p-MLKL to detect oral neoplastic lesion in oral cavity

As described in the above I, the p-MLKL appears in the upper layer of the squamous epithelial tissue, so it is possible to detect the p-MLKL in the upper layer directly in the oral cavity of the subject without collecting the tissue or sample. The p-MLKL in the upper layer may be detected using the immunostaining as in the above I.

However, since fixation with the fixative solution is not possible in this case, after cleaning the oral cavity, the anti-p-MLKL antibody labeled with the labeling substance such as the fluorescent substance, peroxidase, alkaline phosphatase and luciferase is applied directly to the area of the oral cavity intended to be examined (also referred to as an area of interest), and after washing, the signal of the labeling substance is detected. When the labeling substance is the fluorescent substance, the fluorescent signal is detected. When the peroxidase or alkaline phosphatase is used as the labeling substance, the chemiluminescent substrate is used to detect a luminescent signal. The signal may be detected using a bioimaging analyzer or the like.

### III. Test reagent and test kit for use in detection method in vitro

One embodiment of the present invention relates to a test reagent for detecting the p-MLKL used in the method for detecting the oral neoplastic lesion, as described in the above I.

The test reagent for detecting the p-MLKL comprises one or more kinds of anti-p-MLKL antibodies (for example, primary antibody) capable of binding to at least a portion of the p-MLKL. Any of a polyclonal antibody, monoclonal antibody and fragment thereof (for example, Fab, F(ab'), F(ab)₂, etc.) may be used as the “antibody”. The class and subclass of an immunoglobulin of the antibody is not particularly limited. Said antibody may be one screened from an antibody library, and may be a chimeric antibody, an scFv, or the like. It is preferred that the anti-p-MLKL antibody binds, for example, to the MLKL in which the Ser 358 or Ser 345 is phosphorylated. The commercially available antibody described in the above I. may also be used as the anti-p-MLKL antibody.

In addition, the antibody need not necessarily be purified, and an antiserum containing the antibody, ascites, an immunoglobulin fraction fractionated from these, or the like may be used.

The antibody in the test reagent may be in a dried state or dissolved in a buffer such as phosphate-buffered saline. In addition, the test reagent may contain at least one of a stabilizing agent such as B-mercaptoethanol, DTT, or the like; a protective agent such as albumin, or the like; a surfactant such as Polyoxyethylene (20) Sorbitan Monolaurate, Polyoxyethylene (10) Octylphenyl Ether, or the like; and a preservative such as sodium azide, or the like.

The antibody that binds to the p-MLKL may be labeled with the enzyme or fluorescent dye.

The test reagent for detecting the p-MLKL may be provided as a test kit comprising the test reagent and a package insert describing how to use the reagent or the URL of a web page describing how to use the reagent. When the antibody that binds to the p-MLKL is an unlabeled primary antibody, the test kit may also comprise a secondary antibody labeled with the metal nanoparticle, enzyme, or fluorescent dye. In addition, the test kit may comprise the substrate that reacts with said enzyme. Furthermore, the primary antibody comprised in the test kit may be bound to the carrier described in the above I. (ii) as the capture antibody. Furthermore, in the case that the test kit is used for lateral flow immunoassay, the primary antibody, as the capture antibody, together with the secondary antibody, as the detection antibody, may be impregnated into a membrane and dried to be provided as the kit. In this case, a dissolving agent for dissolving the specimen may be included in the kit.

### IV. Test reagent and test kit for use in detection method in vivo

One embodiment of the invention relates to the test reagent for detecting the p-MLKL for use in the method for detecting the oral neoplastic lesion in the oral cavity, as described in the above II.

For an explanation of the test reagent for detecting the p-MLKL, the description in the above III. is hereby incorporated.

The test reagent for detecting the p-MLKL may be provided as the test kit comprising the test reagent and the package insert describing how to use the reagent or the URL of the web page describing how to use the reagent. In addition, when the antibody that binds to the p-MLKL is the unlabeled primary antibody, the test kit may comprise the secondary antibody labeled with the enzyme or fluorescent dye. Furthermore, the test kit may comprise the substrate that reacts with said enzyme.

### V. Photoimmunotherapy, and therapeutic composition for treating oral neoplastic lesion used in photoimmunotherapy

Photoimmunotherapy is a method of killing a target cell by binding a complex of antibody with a photosensitizer to the target cell and irradiating it with light of a predetermined wavelength. The photosensitizer may include, for example, IRdye700DX (IR700) dye, which is a soluble silicon phthalocyanine derivative. The wavelength of the light irradiated is red to near-infrared wavelengths, for example, 680 to 700 nm. The light irradiation may be performed, for example, in the range of 8 to 32 Jules/cm², at about 150 mW/cm².

The therapeutic composition comprises the anti-p-MLKL antibody to which the photosensitizer is bound. For an explanation of the antibody, the description in the above III. is hereby incorporated.

### EXAMPLES

The present invention will be described in more detail below with reference to examples. However, the present invention should not be construed as being limited to the examples.

### I. Materials and Methods

### i. Cases

276 patients who underwent surgical resection in the Department of Otorhinolaryngology, Head and Neck Surgery, at Kansai Medical University Hospital between January 2016 and December 2020 were studied (Table 1). Of the 276 patients, 118 patients had oral squamous cell carcinoma (OSCC) (well-differentiated: 62, moderately-differentiated: 24, poorly-differentiated: 32), 123 patients had precancerous lesion/dysplasia (mild: 35, moderate: 49, severe: 39), and 35 patients had non-neoplastic lesions (hyperplasia: 7, inflammation: 13, normal tissue in the vicinity of the tumor: 15). None of the patients received any treatment before surgery. Histological grades of the tumors were classified according to the WHO grading system.

This study was conducted in accordance with the principles of the Declaration of Helsinki and was approved by the institutional review board of Kansai Medical University Hospital (approval #2020289). In addition, this study was conducted after obtaining informed consent from the patients.

### ii. Preparation of Tissue

For the tissue collected from the patient, a formalin-fixed paraffin-embedded (FFPE) tissue was prepared to produce a tissue microarray (TMA). Sections with a thickness of 4 μm, prepared from a single case, were immunostained for the p-MLKL and tumor areas showing stronger staining intensity were used for analysis. The region that showed stronger staining intensity in the tumor area was marked on the paraffin-embedded block corresponding to the aforementioned section and the region was punched out with a 2 mm biopsy needle. The paraffin block piece obtained by punching was embedded in another paraffin to prepare the TMA. The TMA was thinly sliced to a thickness of 4 μm to prepare a TMA sample.

### III. Immunohistochemical Analysis

The TMA sample was immunostained for the p-MLKL. Immunohistochemical staining of the TMA sample with Anti-MLKL (phospho S358) (Clone ID: EPR9514; catalog number ab187091, RRID: AB_2619685; Abcam; dilution ratio 1: 250) was performed using BOND III fully automated staining system (Leica Biosystems, Melbourne, Australia) according to a standard protocol. Antigen retrieval was performed at pH 6 for 40 minutes at 100° ° C. BOND Epitope Retrieval Solution 1 (#AR9961) citrate-based buffer (pH 6.0) was used as an antigen retrieval solution. BOND Polymer Refine Detection (# DS9800) compact polymer detection system (HRP labeling and DAB coloring) was used as a detection system.

Scoring of the stained samples was performed by two pathologists (Y.N. and K.T.) under a light microscope. Scoring of the p-MLKL was performed in two areas: (i) the upper layer containing the stratum corneum and stratum granulosum or stratum medium, and (ii) the lower layer containing the spinous cell layer and basal cell layer. With respect to distribution of the p-MLKL, for the two regions, distribution area was scored as “≤10%” or “>10%” and staining intensity was scored as 0 (no staining), 1 (mild), 2 (moderate) or 3 (strong). Negative was defined as ≤10% distribution area, or staining intensity of 0 or 1, while positive was defined as >10% distribution area, or staining intensity of 2 or 3. The staining intensity was assessed when positive signals were present in the cell membrane or cytoplasm.

### II. Results

### i. Case of Non-Neoplastic Lesion

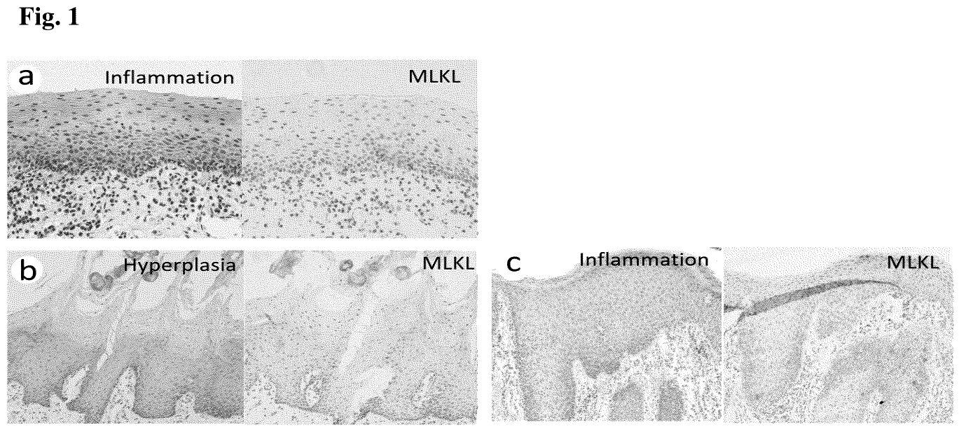

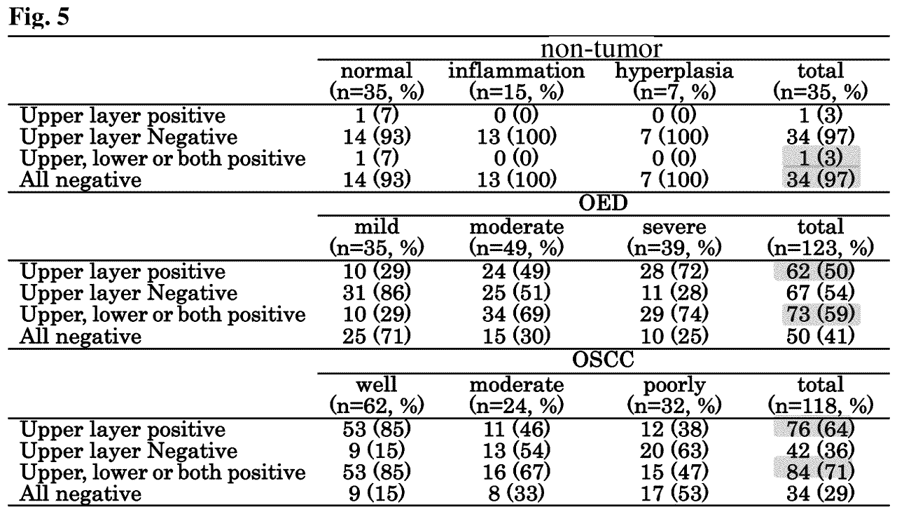

FIG. 1 shows images of an immunostained tissue for the p-MLKL in a case of the non-neoplastic lesion. Panel (a) shows the results in an inflamed tissue. Panel (a) left is an image of an HE-stained tissue, and Panel (a) right is an image of an immunostained tissue for the p-MLKL. Panel (b) shows the results in a hyperplastic tissue. Panel (b) left is an image of an HE-stained tissue, and Panel (b) right is an image of an immunostained tissue for the p-MLKL. As shown in the upper part of FIG. 5, 34 cases (97%) out of a total of 35 cases of non-neoplastic inflammation, hyperplasia and normal were negative. In one case with inflammation, a positive signal for the p-MLKL was observed in the stratum granulosum/stratum intermedium. The results are shown in Panel (c). Panel (c) left is an image of an HE-stained tissue, and Panel 1c right is an image of an immunostained tissue for the p-MLKL.

### ii. Case of Precancerous Lesion

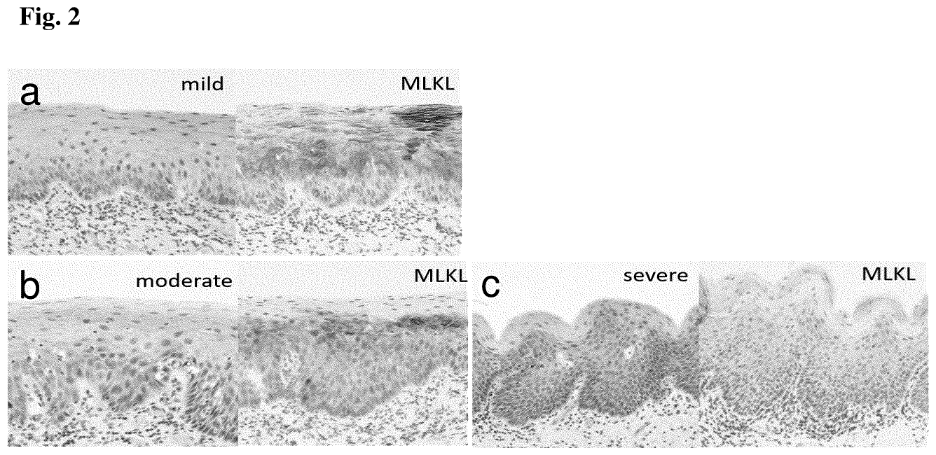

FIG. 2 shows images of an immunostained tissue for the p-MLKL in a precancerous lesion (OED). Panel (a) shows results in a case of the mild dysplasia. Panel (a) left is an image of an HE-stained tissue, and Panel (a) right is an image of an immunostained tissue for the p-MLKL. Panel (b) shows results in a case of the moderate dysplasia. Panel (b) left is an image of an HE-stained tissue, and Panel (b) right is an image of an immunostained tissue for the p-MLKL. Panel (c) shows results in a case of the severe dysplasia. Panel (c) left is an image of an HE-stained tissue, and Panel (c) right is an image of an immunostained tissue for the p-MLKL. As shown in the middle part of FIG. 5, the signal of the p-MLKL was observed in either the upper or lower layer in 73 cases (59%) and in the upper layer in 62 cases (50%).

### iii. Case of Cancer

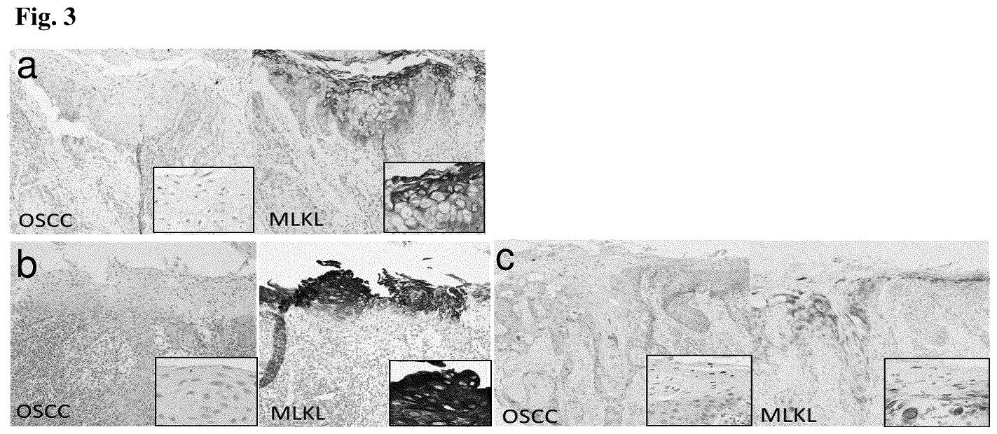

FIG. 3 shows images of an immunostained tissue for the p-MLKL in the squamous cell carcinoma. Panel (a) and Panel (b) show the results of a tissue in which the p-MLKL signal was strongly positive. Panel (a) left and Panel (b) left are images of an HE-stained tissue, and Panel (a) right and Panel (b) right are images of an immunostained tissue for the p-MLKL. Panel (c) shows the results of a tissue in which the p-MLKL signal was moderate. Panel (c) left is an image of an HE-stained tissue, and Panel (c) right is an image of an immunostained tissue for the p-MLKL. As shown in the lower part of FIG. 5, the signal of the p-MLKL was observed in either the upper or lower layer in 84 cases (71%) and in the upper layer in 76 cases (64%).

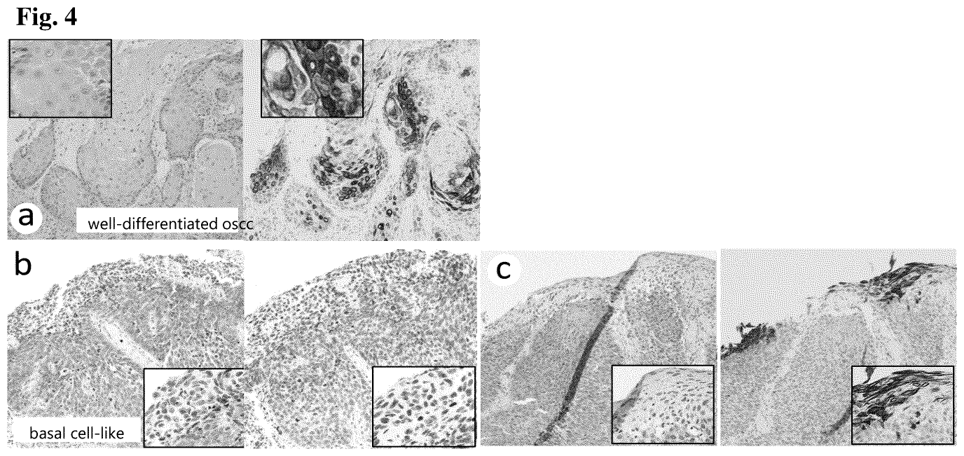

FIG. 4 shows images of an immunostained tissue for the p-MLKL in the squamous cell carcinoma. Panel (a) shows the results of a tissue in a case of well-differentiated squamous cell carcinoma. Panel (a) left is an image of an HE-stained tissue, and Panel (a) right is an image of an immunostained tissue for the p-MLKL. Panel (b) shows results of a tissue in a case of basal cell carcinoma as a subtype of poorly-differentiated squamous cell carcinoma (i.e., in a case of basaloid squamous cell carcinoma). Panel (b) left is an image of an HE-stained tissue, and Panel (b) right is an image of an immunostained tissue for the p-MLKL. Panel (c) shows the results of the tissue in a case of the poorly-differentiated squamous cell carcinoma in which the p-MLKL was positive. Panel (c) left is an image of an HE-stained tissue, and Panel (c) right is an image of an immunostained tissue for the p-MLKL. As shown in the lower part of FIG. 5, the positive signals for the p-MLKL were frequently observed in the well-differentiated OSCC (53 cases, 85%), and there was a tendency for the positive signals to be distributed in the upper layer and keratinocytes.

### iv. Discussion

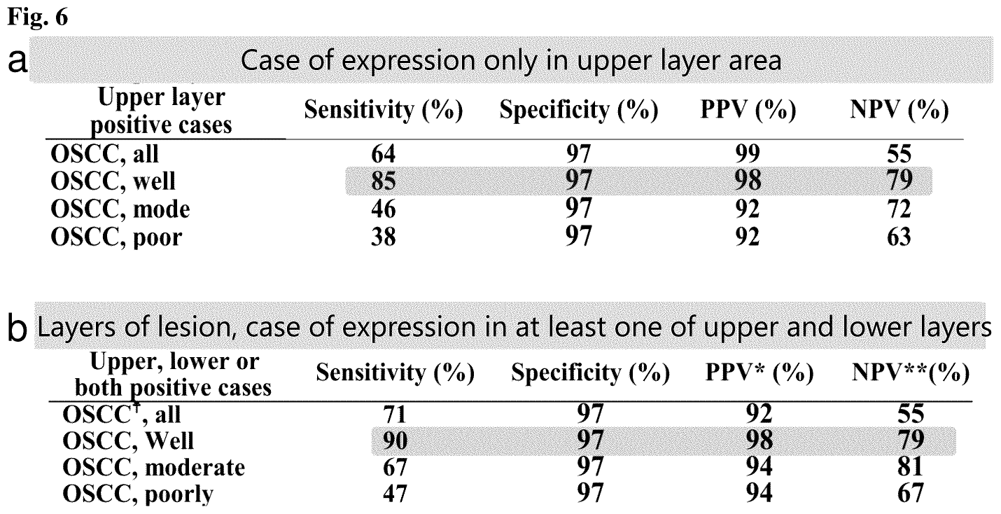

FIG. 6 shows the results of the sensitivity, specificity, positive predictive value (PPV) and negative predictive value (NPV) for the detection of the oral squamous cell carcinoma when the p-MLKL is used. The sensitivity, specificity, PPV, and NPV of the well-differentiated squamous cell carcinoma showed the highest values in terms of test accuracy when the p-MLKL was determined to be positive in the upper layer (Table (a)). From this, it was considered that the detection of the p-MLKL was useful for detecting the well-differentiated squamous cell carcinoma, which is the most difficult to determine whether it is a tumor or not.

The sensitivity, specificity and PPV for detection of the well-differentiated squamous cell carcinoma were 90%, 97% and 98% respectively, when the positive signals were observed in either the upper layer or the lower layer, indicating the possibility that the well-differentiated squamous cell carcinoma may be detected using the p-MLKL as an indicator if the tissue is accurately collected by biopsy or the like.

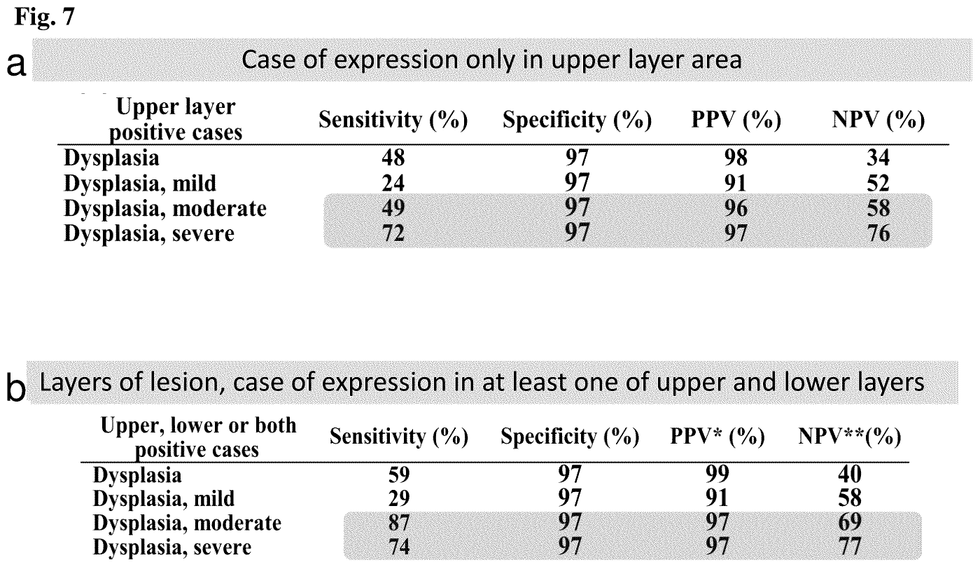

FIG. 7 shows the results of the sensitivity, specificity, PPV and NPV in the precancerous lesion when the p-MLKL is used. Approximately 50% to 70% of the moderate to severe dysplastic tissues requiring treatment were positive in terms of the test accuracy when the p-MLKL was determined to be positive in the upper layer (Table (a)).

When the signal of the p-MLKL was present in either the upper or lower layer, the sensitivity of detection of the moderate to severe dysplasia ranged from 87% to 74% (FIG. 7b). From this, it was considered that the p-MLKL was also useful for detecting the severe dysplastic tissue or cell.

1. A method for detecting an oral neoplastic lesion comprising detecting phosphorylated mixed lineage kinase domain-like protein (MLKL) in an oral tissue or oral-derived cells collected from a subject.

2. The method according to claim 1, wherein the oral neoplastic lesion is a squamous cell carcinoma or dysplasia.

3. The method according to claim 2, wherein the squamous cell carcinoma is a well-differentiated squamous cell carcinoma.

4. The method according to any claim 1, wherein the oral tissue is an upper layer of squamous epithelium or the oral-derived cells are derived from the upper layer.

5. A method for using phosphorylated mixed lineage kinase domain-like protein (MLKL) to detect an oral neoplastic lesion in an oral cavity.

6. A test reagent for use in the method according to claim 1, comprising an anti-phosphorylated mixed lineage kinase domain-like protein (MLKL) antibody.

7. A test kit for use in the method according to claim 1, comprising the test reagent according to claim 6.

8. A test reagent for detecting an oral neoplastic lesion for use in an oral cavity, comprising an anti-phosphorylated mixed lineage kinase domain-like protein (MLKL) antibody.

9. A test kit for detecting an oral neoplastic lesion for use in an oral cavity, comprising the test reagent according to claim 8.

10. A therapeutic composition for treating an oral neoplastic lesion for use in photoimmunotherapy, comprising an anti-phosphorylated mixed lineage kinase domain-like protein (MLKL) antibody.
